(19) United States
(12) Patent Application Publication
(10) Pub. No.: US 2011/0020478 A1
(43) Pub. Date: Jan. 27, 2011

(54) METHOD FOR TREATING NEURAL INJURIES

(76) Inventors: Henrich Cheng, Taipei City (TW); May-Jywan Tsai, Taipei City (TW)

Correspondence Address: OCCHIUTI ROHLICEK & TSAO, LLP, 10 FAWCETT STREET, CAMBRIDGE, MA, 02138, US

(21) Appl. No.: 12/727,344

(22) Filed: Mar. 19, 2010

Related U.S. Application Data
(60) Provisional application No. 61/228,450, filed on Jul. 24, 2009.

(51) Int. Cl. A61K 36/539; A61K 31/7048; A61P 25/00

(52) U.S. Cl. 424/741; 514/27

(57) ABSTRACT

A method for treating neural damage with a composition prepared from roots of bupleurum and scutellaria.

### RELATED APPLICATION

This application claims priority to U.S. Provisional Application No. 61/228,450, filed on Jul. 24, 2009, the content of which is hereby incorporated by reference in its entirety.

### FIELD OF THE INVENTION

The present invention relates to treatment of neural damage with herbal products prepared from bupleurum roots and scutellaria roots.

### BACKGROUND OF THE INVENTION

Nervous system injuries are leading causes of neurologic disability. Spinal cord injury (SCI), a common neural injury, involves damage to the spinal cord that results in loss of sensation and motor control. It can be caused by a disease (e.g., Friedreich's ataxia) or a physical trauma (e.g., contusion) on the spinal cord. Like many other types of neural injuries, SCI often lead to disability and even death due to lack of effective therapy.

Xiao-chai-hu-tang (Sho-saiko-to in Japanese) is a well known herbal composition made from seven medical herbs, i.e., bupleurum (root), scutellaria (root), pinellia (tuber), jujube (fruit), ginseng (root), glycyrrhiza (root), and ginger (rhizome). This composition contains several bioactive ingredients, including saikosaponins, baicalin, baicalein, and glycyrrhizic acid. See Ohtake et al., J. Chromatography B 812 (2004): 135-148. It has been widely used for treating respiratory, hepatobiliary, and gastrointestinal diseases.

### BRIEF SUMMARY OF THE INVENTION

The present invention is based on the unexpected discovery that an herbal composition prepared from the roots of bupleurum and scutellaria exhibits neuro-protective effects and improves functional recovery in SCI rats.

Accordingly, one aspect of the present invention relates to a method for treating neural damage by administering to a subject in need thereof an effective amount of a composition containing an aqueous extract of bupleurum roots (e.g., the roots of Bupleurum scorzonerifolfium Wild) and an aqueous extract of scutellaria roots (e.g., the roots of Scutellaria baicalensis Georgi). The weight ratio between the bupleurum roots and the scutellaria roots can be 7:3. The composition used in this method can contain baicalin, baicalein, wogonin, wogonoside, saikosaponin A, saikosaponin C, and saikosaponin D. In one example, the composition is prepared by extracting the roots of bupleurum and scutellaria, in combination, with water to form a mixture, filtering the mixture to obtain a solution, and lyophilizing the solution to produce a powder. The powder can be dissolved in saline immediately before being administered to a subject in need of the treatment, such as a subject suffering from spinal cord injury (SCI), traumatic brain injury, peripheral nerve injury, Parkinson's disease, Huntington's disease, Alzheimer's disease, amyotrophic lateral sclerosis (ALS), epileptic seizure, or brain ischemia.

The term “treating” as used herein refers to the application or administration of a composition including one or more active agents to a subject, who has neural damage, a symptom of the damage, or a predisposition toward the damage, with the purpose to cure, heal, alleviate, relieve, alter, remedy, ameliorate, improve, or affect the damage, the symptoms of the damage, or the predisposition toward the damage. “An effective amount” as used herein refers to the amount of each active agent required to confer therapeutic effect on the subject, either alone or in combination with one or more other active agents. In one example, the effective amount of the above-described composition is 20 mg per kg of a subject's body weight. Effective amounts vary, as recognized by those skilled in the art, depending on route of administration, excipient choice, and co-usage with other active agents.

Another aspect of this invention relates to the use of the composition described above for the manufacture of a medicament for treating neural damage.

The details of one or more embodiments of the invention are set forth in the description below. Other features or advantages of the present invention will be apparent from the following drawings and detailed description of several embodiments, and also from the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1(A) and (B) shows the HPLC fingerprint spectra of HD2 at (A) 203 and (B) 280 nm, respectively.

FIG. 2 shows the effects of HD2 on H₂O₂- and t-BOOH-induced free radical formation in microglia cultures. The symbols “#” and “**” indicate statistical significance by one way ANOVA and Bonferroni t-test at p<0.01 (H₂O₂ or t-BOOH treated cells vs. control) and p<0.01 (H₂O₂ plus HD2 vs. H₂O₂ alone or tBOOH plus HD2 vs. tBOOH alone), respectively

FIG. 3 shows the effects of HD2 and baicalein on H₂O₂-induced free radical formation in (A) cortical mixed neuronal-glial cultures and (B) spinal cord mixed neuronal-glial cultures. The symbols “HD2 20”, “HD2 40”, “HD2 80” and “HD2 200” refer to the HD2 at 20, 40, 80 and 200 μg/ml, respectively; “bai 2” and “bai 10” refer to baicalein at 2 μM and 10 μM, respectively; and “#” and “**” indicate statistical significance by one way ANOVA and Bonferroni t-test at P<0.05 (H₂O₂ treated cells vs. control) and P<0.01 (HD2 and H₂O₂ treated cells vs. H₂O₂ treated cells), respectively.

FIG. 4 shows the effects of HD2 and baicalein on t-BOOH-induced free radical formation in (A) cortical mixed neuronal-glial cultures and (B) spinal cord mixed neuronal-glial cultures. The symbols “HD2 20”, “HD2 40”, “HD2 80” and “HD2 200” refer to HD2 at 20, 40, 80 and 200 μg/ml, respectively; “bai 2” and “bai 10” refer to baicalein at 2 μM and 10 μM, respectively; and “#” and “**” indicate statistical significance by one way ANOVA and Bonferroni t-test at P<0.05 (t-BOOH treated cells vs. control) and P<0.01 (HD2 and t-BOOH treated cells compared with t-BOOH treated cells), respectively

FIG. 5 shows the effect of HD2 on lipopolysaccharide (LPS)-induced toxicity in spinal cord mixed neuronal-glial cultures wherein (A) shows the number of iNOS-positive cells and (B) shows the amount of nitrite release, in each group of cells. The symbols “*” and “**” indicate statistical significance by one way ANOVA and Bonferroni t-test at P<0.05 (HD2 vs. saline or HD2 and LPS-treated vs. LPS-treated cells) and P<0.01 (HD2 and LPS-treated vs. saline and LPS-treated cells), respectively.

FIG. 6 shows the effect of HD2 on LPS stimulation in mesencephalic mixed neuronal-glial cultures wherein (A) shows the number of iNOS-positive cells and (B) shows the amount of nitrite released, in each group of cells. The symbols “*” and “* *” indicate statistical significance by one way ANOVA and Bonferroni t-test at P<0.05 (HD2 and LPS-treated vs. saline and LPS-treated cells) and P<0.01 (HD2 and LPS-treated vs. saline and LPS-treated cells), respectively.

FIG. 7 shows the result of the evaluation of the hind limb function in contusive SCI rats. The symbol “*” indicate statistical significance by one way ANOVA and Bonferroni t-test at P<0.05 (HD2-treated SCI vs. SCI group).

FIG. 8 shows the effect of intraperitoneal administration of HD2 in contusive SCI rats, wherein (A) refers to the lactate level in the rats and (B) refers to the malondialdhyde (MDA) level in the rats; and (C) and (D) refer to the ED-1 expression level in the rats. The symbols “#” and “*” indicate statistical significance by one way ANOVA and Student-Newman-Keuls Method at P<0.05 (SCI vs. sham) and P<0.05 (SCI vs. HD2-treated SCI), respectively.

### DETAILED DESCRIPTION OF THE INVENTION

Unless defined otherwise, all technical and scientific terms used herein have the same meanings as is commonly understood by one of skill in the art to which this invention belongs. All publications and patents referred to herein are incorporated by reference.

As used herein, the articles “a” and “an” refer to one or more than one (i.e., at least one) of the grammatical object of the article. By way of example, “an element” means one element or more than one element.

The present invention relates to a method for treating neural damage with an herbal composition prepared from bupleurum roots and scutellaria roots. This composition can be prepared by extracting the roots of bupleurum and scutellaria, either in combination or separately, with water (i.e., pure water or a suitable water-containing solvent). See, e.g., Wang et al., Int. J. Mol. Med. 2005, 16(2):221-231 and Lee et al., Liver Int. 2008, 28(6): 841-855).

In one example, the composition used in the method of this invention is prepared as follows. Bupleurum roots and scutellaria roots are mixed at a suitable weight ratio, dispersed in hot water, and incubated at a temperature ranging from about 95 to about 100° C. for a sufficient period. The resultant mixture is then filtered to remove water-insoluble substances and the water-soluble fraction is harvested and lyophilized to yield a powder. The ingredients in this powder can be identified via conventional methods, e.g., HPLC.

Preferably, the composition described herein includes one or more of baicalin (molecular wt. 446.37), baicalein (molecular wt. 270.20), wogonin (molecular wt. 284.27), wogonoside (molecular wt. 463.27), saikosaponin A (molecular wt. 780.99), saikosaponin C (molecular wt. 927.14), and saikosaponin D (molecular wt. 780.99). Baicalein is a baicalin aglycone, while wogonin is a wogonoside aglycone. Their structures are as follows.

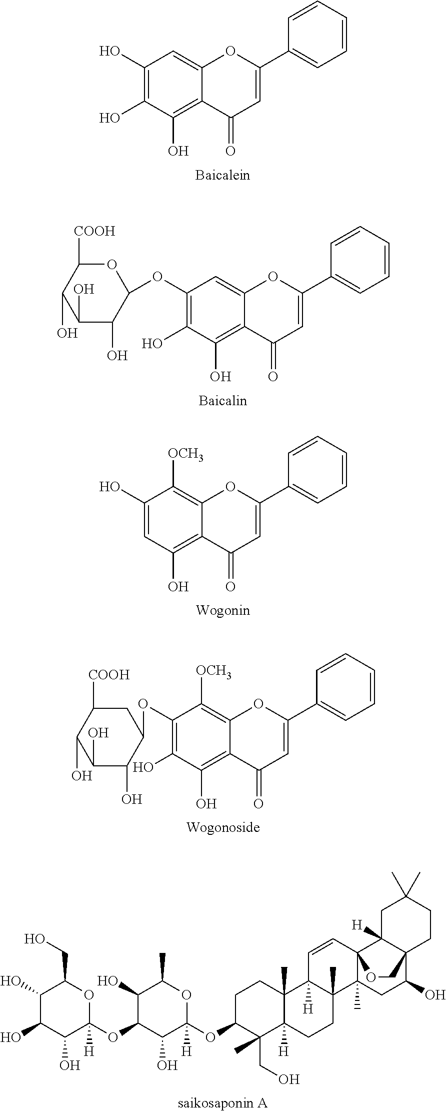

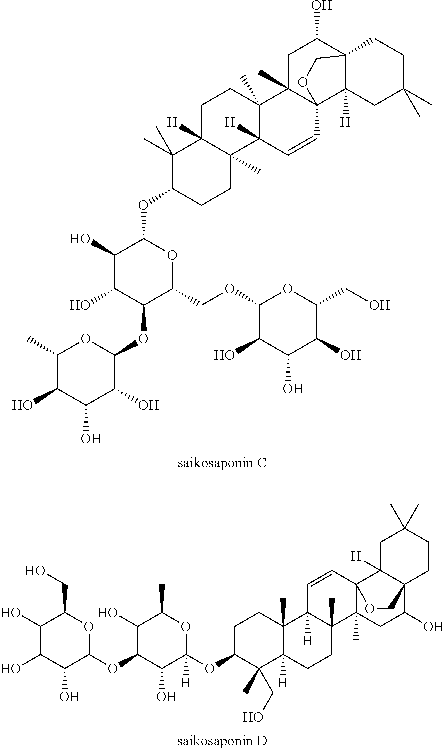

The composition described above can be used for treating a subject afflicted with damage to a nervous system.

As used herein, the phrase “damage to a nervous system” refers to impairment or loss of function of one part of the nervous system of a subject. The damage may be caused by trauma as a result of, for example, car accidents, falls, blow, gunshot, sports injuries, and war injuries, or by a disease such as tumor, a neurodegenerative disease (e.g., Parkinson's disease, Alzheimer's disease. Huntington's disease, and amyotrophic lateral sclerosis (ALS)), or other diseases (e.g., demyelination, ischemia, and epileptic seizure). In one example, the damage occurs in the central nervous system, e.g., in the spinal cord.

The most common type of spinal cord injury (SCI) is caused by contusion injury (contusive SCI), which is induced by bruising of the spinal cord. Other types of injuries include lacerations, e.g., severing or tearing of nerve fibers such as damage caused by a gunshot wound. Severe SCI often causes paralysis, i.e. loss of control of voluntary movements and muscles of the body, and loss of sensation and reflex function below the point of injury, including autonomic activity and other activities such as bowel and bladder control. Acute SCI contusion also produce a variety of pathophysiological conditions such as inflammation, fiber deformation, increased vascular permeability, local ischemia, intraneuronal edema and local demylination.

A subject in need of the treatment disclosed herein includes humans and non-human vertebrates that suffer from neural damage or a symptom thereof. Non-human vertebrates include mammals, birds, lizards, frogs. Examples include, but are not limited to, cats, dogs, cattle, horses, sheep, goats, and swine.

Typically, damage to a nervous system induces formation of free radicals and microglial activation in the damaged tissue of the nervous system, and the composition of the invention is useful as an antioxidative and anti-inflammatory agent to reduce the level of the free radicals and inflammation. More typically, damage to a nervous system results in death of cells of the nervous system, and the composition of the invention can inhibit the death of the cells such as neuronal and/or glial cells.

In addition, the composition is preferably in a form of lyophilized powder which is freshly dissolved in saline before administration to the subject.

The herbal composition used in the method of this invention may be administered to a subject via any suitable route such as orally, parentally (e.g. intramuscularly, intravenously, subcutaneously, interperitoneally), transdermally, rectally, by inhalation and the like. To facilitate administration, it is preferably formulated into a pharmaceutical composition with a pharmaceutically acceptable carrier. Such carriers include, but are not limited to, saline, buffered saline, dextrose, water, glycerol, ethanol and combinations thereof.

To facilitate delivery, the composition can be formulated into a pharmaceutical composition with a suitable pharmaceutically acceptable carrier. The term “pharmaceutically acceptable carrier” as used herein refers to a carrier that is compatible with the active ingredient of the composition, and preferably, capable of stabilizing the active ingredient and not deleterious to the subject to be treated. Exemplary carriers include, but are not limited to, saline, buffered saline, dextrose, water, glycerol, ethanol and combinations thereof.

The herbal composition or the pharmaceutical composition thereof may be prepared or constituted into any form suitable for the mode of administration selected. For example, forms suitable for oral administration include solid forms, such as pills, capsules, granules, tablets, and powders, and liquid forms, such as solutions, syrups, elixirs, and suspensions. Forms useful for parenteral administration include solutions, emulsions, and suspensions.

In one embodiment of the present invention, the herbal composition is administered by intraperitoneal (ip) injection. Preferably, the herbal extract, in a form of lyophilized powder, is freshly dissolved in saline before administration to the subject.

The amount of the herbal composition to be administered to a subject varies in view of many parameters, such as the conditions of the subject and the type and severity of the damage. Practitioners skilled in the art will readily determined the suitable amount of the herbal composition used in the method of this invention via routine experimentation. A suitable amount of the herbal composition of the invention, when applied to the subject suffering from SCI, for example, attains a desired effect, for example, repairing injured area and/or enhancing at least partially functional restoration of the injured spinal cord. In a preferred embodiment of the present invention, the amount of the herbal composition to be administered is more than about 2 mg/kg. More preferably, the herbal composition is administered to the area of injury in an amount of 20 mg/kg daily by ip injection for at least 7 consecutive days.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

### EXAMPLES

### Chemicals

Unless stated otherwise, all other chemicals were purchased from Sigma-Aldrich Co (St. Louis, Mo.).

### Example 1

### Preparation and Characterization of the Herbal Composition

An aqueous herb herbal composition of bupleurum and scutellaria, i.e., HD2, was prepared based on the methods described in Wang et al., (2005) and Lee et al. (2008) supra. Briefly, the roots of S. baicalensis Georgi (36 g) and the roots of B. scorzonerifolfium Wild (84 g) were boiled in 3600 ml of water at 100° C. until the total volume reduced to 1000 ml. The extracts were then filtered through layers of gauze, and the filtrate was collected and lyophilized. The yield of the lyophilized powder was about 16 gm.

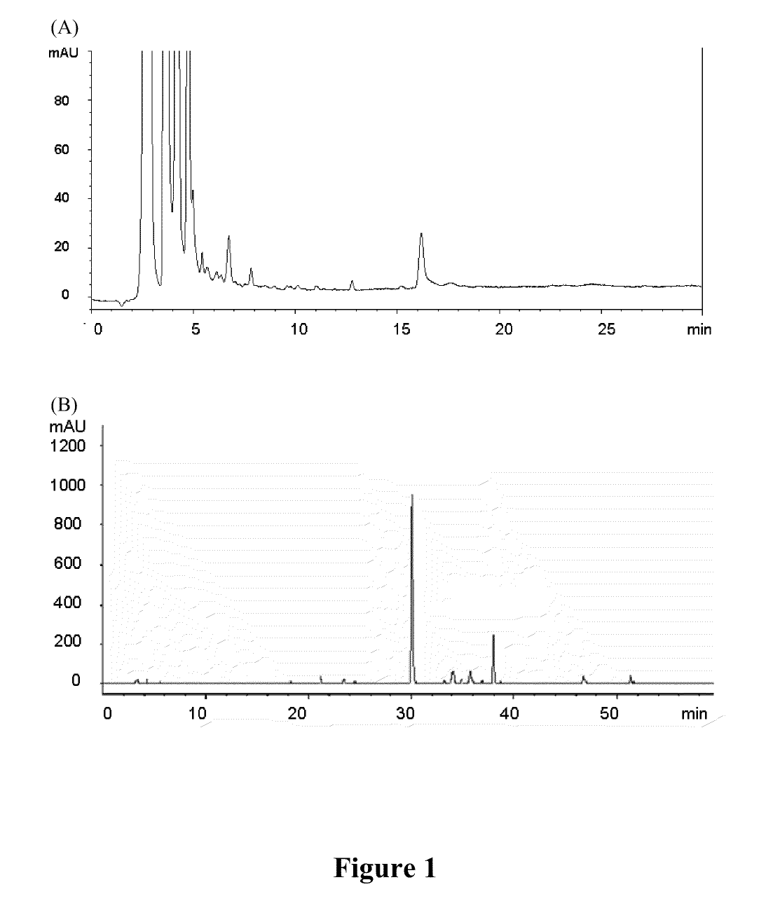

To identify the ingredients of the HD2, a high performance liquid chromatography (HPLC) analysis was conducted based on the method described in Lee et al. (2008). Briefly, the HPLC analysis was performed with a stationary phase (Cosmosil 5 C18-MS-II column) and a mobile phase (10 mM monosodium phosphoric acid-acetonitrile; 69:31, v/v, pH 3.0), at a flow rate of 1 ml/min. The UV detector wavelengths were set at 203 nm and 280 nm. FIGS. 1(A) and (B) show the result of the fingerprint spectra at 203 and 280 nm, respectively.

FIG. 1(A) exhibits the peaks of saikosaponin A, saikosaponin C, and saikosaponin D at the retention time ranging from 9 to 23 min. FIG. 1(B) exhibits the peaks of baicalein, baicalin wogonin and wogonoside at the retention time of 47, 30, 39 and 51 min, respectively. The HD2 was subsequently confirmed to include the following ingredients: baicalin (molecular wt. 446.37) and its aglycone baicalein (molecular wt. 270.20), wogonoside (molecular wt. 460.27) and its aglycone wogonin (molecular wt. 284.27), saikosaponin A (molecular wt. 780.99), saikosaponin C (molecular wt. 927.14) and saikosaponin D (molecular wt. 780.99).

The HD2 in the form of lyophilized powder was freshly dissolved in saline for the following in vitro culture experiments and in vivo administration.

### Example 2

### HD2 performed as an antioxidant in cells of the nervous system

1. Microglia Cells

It is known that generation of free radical plays an important role in pathophysiological development of nervous system injury, and a variety of free radical scavengers have been suggested to have therapeutic potential for protecting the injured nervous system. For reviews, see Kwon et al. Spine J. 4: 451-464 and Wang et al. (2006), Curr. Pharm. Des. 12(27):3521-33.

In the present example, oxidative stress was induced by treating microglia cells with free radical generators, i.e., H₂O₂ or t-BOOH, and the antioxidant activity of HD2 was examined by a dichlorofluorescein (DCF) assay using a fluorescent microplate reader.

2′,7′-dichlorodihydrofluorescein diacetate (DCFH-DA, Molecular Probe D-399; Invitrogen, CA), a sensitive and widely used probe for detection of intracellular oxidant production, was used to detect the formation of intracellular reactive oxygen species (ROS). DCFH-DA can freely enters the cell and becomes modified by intracellular esterases into DCFH, a hydrophilic (and hence “trapped”), nonfluorescent reporter molecule. Oxidation of DCFH creates a highly fluorescent molecule, DCF, which can be detected by fluorescence plate reader at the excitation/emission of 485 nm/538 nm.

Microglial cells were purified from rat mixed glial cultures (Tzeng and Huang, J. Cell Biochem. 90(2) (2003): 227-33). Briefly, after culture for 10 to 14 days, floating cells and weakly attached cells on the mixed glial cell layer were isolated by shaking the flask. The resulting cell suspension was transferred to multiwell plates (Corning, USA) and allowed to adhere at 37° C. After 30 mins, unattached cells were discarded and the microglial cells were collected as strongly adhering cells. The purity of the microglial cells was more than 96% which was determined by immunostaining for ionized calcium-binding adapter molecule-1 (IBA-1, Wako Chemicals, Japan) or the macrophage marker, ED-1 (Serotec, UK).

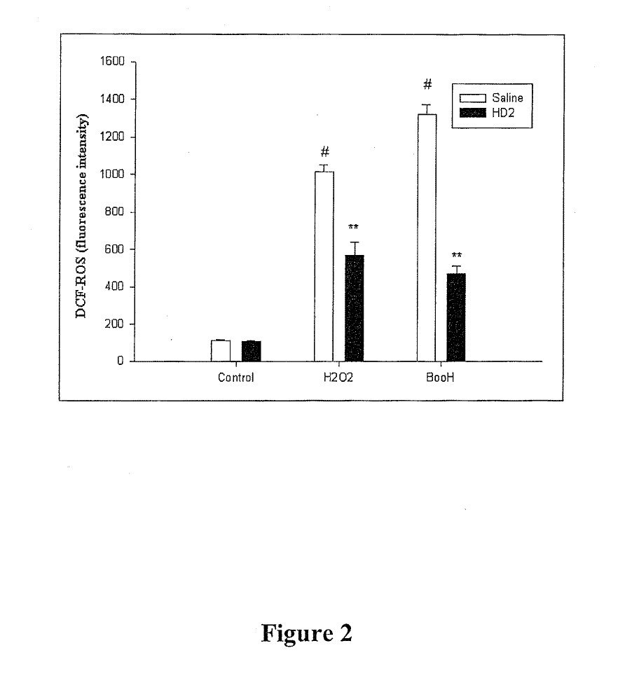

The microglial cells were preloaded with 50 μM of DCFH-DA for 1 hr. The medium was then replaced with growth medium containing H₂O₂ (1 mM) or t-BOOH (0.75 mM) in the presence or absence of HD2 (80 μg/ml). Two hours later, the cellular DCR-ROS level was measured by a fluorescence plate reader. Data are expressed as means±SEM from 3 independent experiments done in triplicate. FIG. 2 shows the results of the DCR-ROS level in the cells.

As shown in FIG. 2, the two free radical generators, H₂O₂ or t-BOOH, significantly increased the level of DCF-ROS in the microglial cells (P<0.01), and HD2 significantly lowered the toxin-induced DCF-ROS level in the cells (P<0.01, H₂O₂ plus HD2 vs. H₂O₂ alone or tBOOH plus HD2 vs. tBOOH alone).

2. Mixed Neuronal-Glial Cultures

Mixed neuron-glia cell cultures were prepared from the spinal, mesencephalic and cortical regions of an embryonic Sprague-Dawley rat fetus (gestation day 15), respectively, as described in Tsai et al. (Ann. N.Y. Acad. Sci. 1042 (2005):338-348). Briefly, cells were dissociated with a mixture of papain/protease/deoxyribonuclease I (0.1%:0.1%:0.03%) and plated onto poly-lysine coated dishes at a density of 1 to 2×10⁵ cells/cm² with Dulbecco's modified Eagle's medium (DMEM) supplemented with 10% fetal bovine serum (FBS). On the second day after cell seeding, the cultures were maintained in DMEM+10% FBS or in DMEM supplemented with N2 (Invitrogen, Carlsbad, Calif.) as serum-free medium.

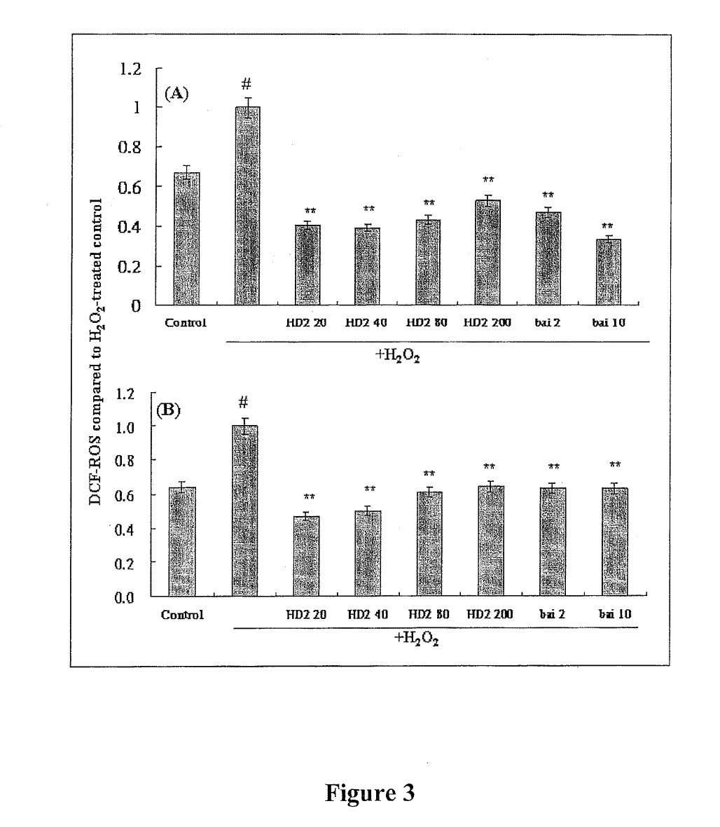

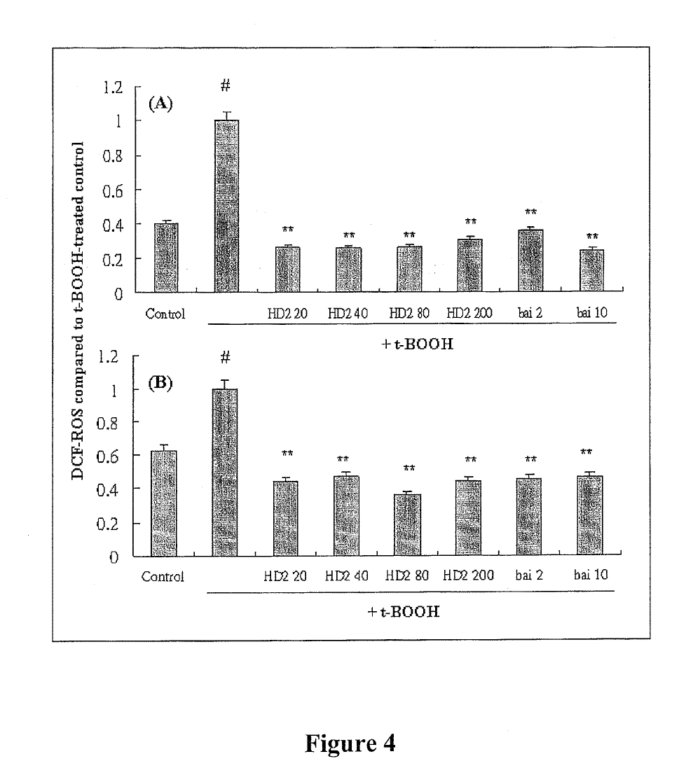

On the 2nd or 3rd day after cell seeding, the cultures were firstly loaded with 50 μM DCFH-DA in serum-free medium (DMEM+N2) for 1 hr. The medium was then replaced with growth medium containing H₂O₂ (1 mM) or t-BOOH (0.75 mM) and the cultures were maintained in the growth medium for 2 hrs. HD2 (20 to 200 μg/ml) and baicalein (2 or 10 μM) were then added to the cultures within 10 minutes after the toxin treatment started, respectively. The resulted fluorescent DCF levels were measured by a fluorescence plate reader (ex/em: 485 nm/538 nm). FIGS. 3 and 4 shows the results of the measurement.

As shown in FIGS. 3(A) and (B), H₂O₂ at 1 mM significantly increased DCF-ROS level in both of the mixed neuron-glial cultures (the cortical regions and the spinal cord); and HD2 (20 to 200 μg/ml) significantly inhibited the free radical level induced by H₂O₂ in both of the cultures (P<0.01). Concurrently, one major component of the HD2, baicalein at 2 and 10 μM, significantly reduced the free radical level induced by H₂O₂ in both cultures.

Similarly, as shown in FIGS. 4 (A) and (B), t-BOOH at 0.75 mM significantly increased DCF-ROS level in both of the mixed neuron-glial cultures (the cortical regions and the spinal cord); and HD2 (20 to 200 μg/ml) and baicalein (2 or 10 μM) significantly inhibited the free radical level induced by t-BOOH in both of the cultures (P<0.01).

Accordingly, HD2 have been proved in the present study for the first time to have a significant effect on reducing toxin-induced free radical formation in cells of the nervous system, and it is thus suggested that HD2 can be used as an antioxidant to treat damage in association with free radical formation in the nervous system.

### Example 3

### HD2 Inhibited Toxin-Induced ATP Depletion and Cell Death in Mixed Glial Cells

Mixed glial cultures were prepared from cerebral cortex of newborn Spraque-Dawley (SD) rats as described in Tsai and Lee (Free Radical Biology & Medicine 24 (1998): 705-713). Briefly, triturated cortex or spinal cords were passed through nylon clothes (80 and 10 μm), plated in 75 cm² flasks, and maintained in DMEM containing 5.5 mM glucose and supplemented with 10% fetal calf serum (FCS). The cells were incubated at 37° C. in a water-saturated atmosphere of 5% CO₂/95% air. To free cultures from contaminated cells, cultures were purified on the 10^{th} day by shaking overnight at 180 rpm to remove the suspended cells. The cultures in the flasks were replated into multiwell plates. The cultures were subsequently confirmed to exhibit greater than 90% positive staining for the glial fibrillary acidic protein (GFAP), an astroglial marker.

1. ATP Measurement

Intracellular ATP level was measured from the perchloric acid (PCA) extracts of the mixed glial cultures by a luciferin-luciferase kit (Sigma-Aldrich). The assay was based on quantitative measurement of luminescent light produced as a result of an enzyme reaction catalyzed by firefly luciferase. Briefly, the mixed glial cultures were treated with H₂O₂ (500 μM) or t-BOOH (200 μM) in the presence or absence of HD2 (10 μg/ml) for 17 hrs. Before obvious cell damage occurred, cultures were harvested for ATP measurement. Table 1 shows the result of the ATP measurement.

TABLE 1

| | ATP (M) | | |
|---|---|---|---|
| Treatment | Control | H₂O₂ (500 M) | t-BOOH (200 M) |
| Saline | 31 | 16 | 16 |
| HD2 10 g/ml | 32 | 33 | 33 |

As shown in Table 1, H₂O₂ or t-BOOH reduced ATP level in the mixed glial cells (due to a lower synthesis of ATP in cells resulted from impairment of oxidative phosphorylation), and HD2 at a dosage of 10 μg/ml successfully inhibited the toxin-induced ATP depletion in the cells.

2. Cell Death Analysis (LDH Assay)

A lactate dehydrogenase (LDH) assay was conducted to determine loss of cell viability of the mixed glial cultures. Briefly, the mixed glial cultures were treated with H₂O₂ (500 μM) for a prolonged period of time (24 hrs), and the level of LDH released from the cells was determined. A higher LDH level indicates a higher proportion of cell death. Table 2 shows the result of the LDH assay.

TABLE 2

| | LDH release | |
|---|---|---|
| Glial | (Saline-Control %) | |
| Treatment | Control | H₂O₂ (500 uM) |
| Saline | 100.0 ± 6.0 | 256.4 ± 7.5# |
| HD2 10 g/ml | 99.1 ± 4.9 | 146.5 ± 5.8** |

#p < 0.01 (H₂O₂ vs. control);
**p < 0.01 (HD2 plus H₂O₂ vs. H₂O₂).

As shown in Table 2, H₂O₂ induced a significant loss of cell viability, as indicated by LDH release, in the mixed glial cultures (p<0.01, H₂O₂ vs. control); and HD2 (10 μg/ml) significantly inhibited H₂O₂-induced LDH release (P<0.01).

Table 1 and 2 also suggest that oxidative phosphorylation is impaired by H₂O₂ treatment prior to the onset of cytotoxicity.

### Example 4

### HD2 Inhibited Toxin-Induced Cell Death in Mixed Neuronal/Glial Cells

The protective effect of HD2 was further examined in mixed neuronal/glial cultures isolated from the cortical, mesencephalic and spinal cord regions of rat fetuses, respectively.

Mixed neuron-glia cell cultures were prepared as described in Example 2. The cultures were treated with H₂O₂ (500 μM) in the presence or absence of HD2 (10 μg/ml) for 24 hrs, and then the level of LDH released from the cells was determined. Table 3 shows the result of the LDH assay.

TABLE 3

| | LDH release (saline-control %) | | |
|---|---|---|---|
| Cells | | Control | H₂O₂ (500 M) |
| Cells from cortical neurons | Saline | 100 | 136.5 |
| | HD2 | 86.5 | 66.7 |
| Cells from mesencephalic neurons | Saline | 100 | 132.2 |
| | HD2 | 90.2 | 103.4 |
| Cells from spinal cord neurons | Saline | 100 | 183.1 |
| | HD2 | 95.8 | 152.8 |

Data shown in the table are means from two independent experiments done in duplicate.

As shown in Table 3, H₂O₂ induced a loss of cell viability, as indicated by LDH release, in all of the H₂O₂-treated cultures and HD2 (10 μg/ml) effectively inhibited H₂O₂-induced cell death in the cortical, mesencephalic and spinal cultures.

### Example 5

### HD2 Protected Mixed Neuronal-Glial Cultures of Spinal Cord from LPS- and Kainate-Induced Toxicity

Mixed neuronal-glial cultures from the spinal cord of a rat were prepared as described in Example 2. On the second day after cell seeding, the mixed neuronal-glial cultures from the spinal cord were treated with HD2 (10 μg/ml) in the presence or absence of endotoxin, lipopolysaccharide (LPS, 1.2 μg/ml, E. coli 0111:B4), or excitotoxin, kainic acid (KA, 150 μM) for 2 days. The cells were harvested for an immunohistochemistry assay, and the medium was collected for determining the release of nitrate/nitrite.

For the immunohistochemistry assay, cells, after paraformaldehyde fixation and triton X-100 permeabilization, were incubated with the primary antibodies of anti-betaIII tubulin (covance), anti-cyclooxygenase-2 (COX-2; cayman) or inducible nitric oxide synthase (iNOS; BD transduction) overnight (4° C.), and subsequently incubated with respective secondary antibodies conjugated with fluorophore 488 or cy3 at room temperature for 90 mins. The cells were then observed under a microscope and counted for quantification.

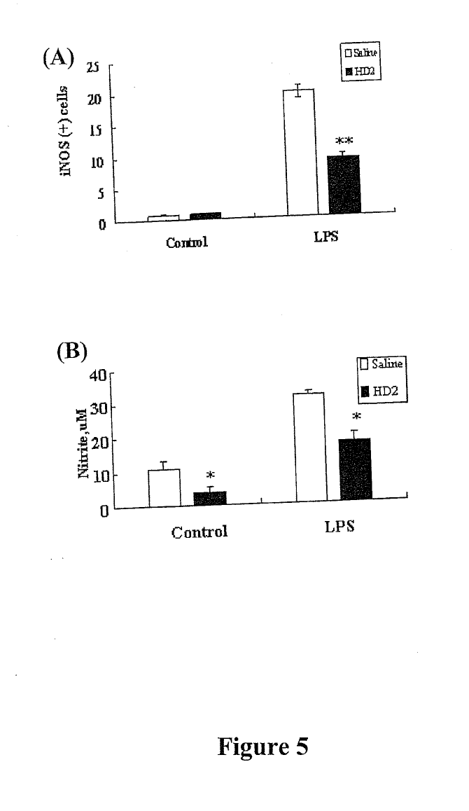

According to the results (data not shown), HD2 enhanced the survival of the mixed neuronal-glial cells of spinal cord, and HD2 inhibited KA-induced cell damage and COX-2 expression in the mixed neuron-glial cultures. In addition, according to FIG. 5 (A), HD2 reduced the number of LPS-induced iNOS positive cells in the mixed neuron-glial cultures.

On the other hand, the production of nitric oxide (NO) was assayed as accumulation of nitrite in medium using colorimetric reaction with Griess reagent. Briefly, after 2 days of LPS treatment, the culture supernatants (150 μl) were collected, mixed with 50 μl of Griess reagent containing 1% sulfanilaminde, 0.1% naphthyl ethylene diamine dihydrochloride, and 2% phosphoric acid, and incubated at room temperature for 10 mins. The absorbance was measured at 540 nm. Sodium nitrite (NaNO₂) was used as the standard to calculate the amount of nitrogen dioxide (NO2). FIG. 5 (B) shows the results of the measurement.

According to FIG. 5(B), LPS induced release of nitric oxide (NO) as shown by nitrite level in the mixed neuronal-glial cells; and HD2 significantly reduced LPS stimulation in the cells.

### Example 6

### HD2 Enhanced Dopaminergic Neuronal Survival and Reduced LPS Stimulation in Mixed Neuronal-Glial Cultures of Mesencephalic Regions

Mixed neuronal-glial cultures of mesencephalic regions of rat fetal brains were prepared as described in Example 2. On the second day after cell seeding, cultures in 24-multiwell plates were treated with HD2 (10 μg/ml). The cultures were then incubated for 5 days with medium refilled and HD2 replenished on the 3^{rd} day. Cultures were then fixed and processed for an immunohistochemical analysis with antibodies against the marker of dopaminergic neurons, i.e. anti-tyrosine hydroxylase (TH). Cells with a positive signal were counted (1.5 mm² per well). Table 4 shows the result of the cell counting. Data are expressed as means±SEM from 3 independent experiments done in duplicate.

TABLE 4

| Treatment | TH(+) cells/well |
|---|---|
| Control | 27.3 ± 3.5 |
| HD2 10 g/ml | 67.7 ± 8.7** |

**P < 0.01 by one way ANOVA

As shown in Table 4, HD2 significantly enhanced the survival of dopaminergic neurons (** P<0.01, HD2 vs. control).

In addition, the effect of HD2 on LPS stimulation was examined for the third day cultures. LPS (1.2 μg/ml) was added to the cultures in the presence or absence of HD2 (10 μg/ml). Cultures were then incubated for 2 days. Cells were then fixed for immunoreactive staining with antibodies against IL-β (R&D #AF 501) or iNOS, and the media were collected for measurement of nitrite by Griess assay.

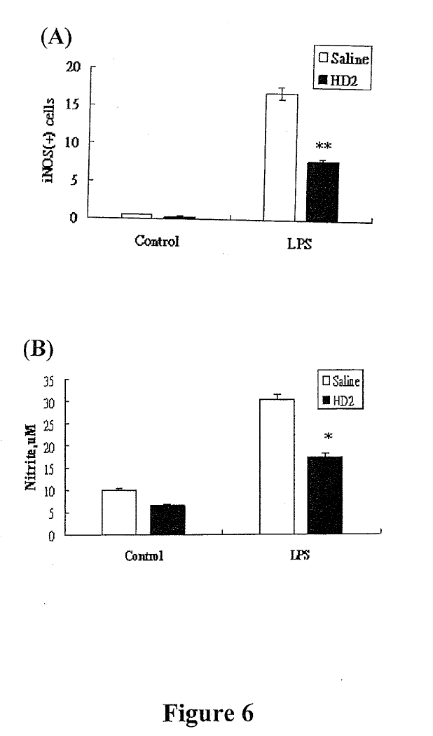

According to the results of the immunoreactive staining (data not shown), HD2 inhibited the number of LPS-stimulated IL-1β (+) cells. In addition, as shown in FIG. 6(A), HD2 inhibited the number of LPS-stimulated iNOS (+) cells. Furthermore, as shown in FIG. 6 (B), HD2 inhibited the LPS-stimulated release of NO as shown by nitrite level.

Accordingly, HD2 has been proved in the present study to have a significant effect on reducing LPS- or KA-induced increase of inflammatory mediators in cells of the nervous system (FIGS. 5 and 6), and it is thus suggested that HD2 can also be used as an anti-inflammatory agent to treat damage in association with inflammation in the nervous system.

### Example 7

### HD2 Increased Cell Proliferation in Subventricular Zone (SVZ) Neuro-Progenitor Cultures

The proliferative activity of neuroprogenitor cells from the SVZ was investigated by treating cells with 5-bromo-2-deoxyuridine (BrdU), a thymidine analog incorporated into genetic material, during the S phase of mitotic division.

Neuroprogenitor cells were isolated from SVZ or spinal cords of neonatal rats, and maintained as neurospheres in DMEM supplemented with B27 (Gibco) and growth factors (EGF/bFGF; 10 ng/ml each) for 2 weeks. The neurospheres were subcultured and equally dispersed in multiwell plates containing DMEM/B27 medium with or without growth factor (EGF/bFGF) supplement. HD2 at various concentrations (25 to 200 μg/ml) were added to the cultures. The cultures were then incubated for 3 days. One hour before cell fixation, cultures were pulsed with BrdU (10 μM, Roche Diagnostics, Germany) for 1 hr to assess mitotic activity according to the method described in Jin et al. (J. Neurochem. 93 (2005): 1251-1261).

After washing with PBS, cells were fixed with 4% paraformaldhyde for 30 mins at room temperature (RT). After washing with PBS, cells were denatured with 2 M HCl for 10 mins at 37° C. Then cells were repeatedly washed with PBS until the pH reached 6.5 or above, and incubated with a blocking solution for 30 mins at RT to prevent nonspecific reactions. Mouse anti-BrdU antibodies (Chemicon, Temecula, Calif.) and rabbit anti-βIII tubulin antibodies were added and incubated overnight at 37° C. After washing three times with PBS, Alexa Fluor 488-labeled donkey antimouse IgG antibodies (Molecular Probes, Eugene, Oreg.) and cy3-labeled donkey anti-rabbit IgG (Jackson lab) antibodies were added and incubated for 45 mins at 37° C. and then cells were washed three times with PBS. Seven random fields (1.5 mm² per view) were captured in each well, and BrdU-positive cells were counted. Table 5 shows the results of the cell counting.

TABLE 5

BrDu(+) cells/1.5 mm²

| | W/O Growth | With Growth |
|---|---|---|
| Treatment | factors | Factors |
| Control | 20 | 31 |
| HD2 25 g/ml | 31.7 ↑ | 37.3 ↑ |
| HD2 50 g/ml | 26.7 ↑ | 48.7 ↑ |
| HD2 100 g/ml | 31.3 ↑ | 39.7 ↑ |
| HD2 200 g/ml | 44.3 ↑ | 62.3 ↑ |

According to Table 5, HD2 (25 to 200 μg/ml) increased cell proliferation (BrdU (+) cells) in SVZ cultures regardless of the presence of growth factors in the culture medium, indicating that HD2 alone can promote progenitor cell proliferation.

### Example 8

### Continuously Intraperitoneal Injection of HD2 Improved Hindlimb Functional Restoration and Reduced Early Microglial Infiltration in Contusive SCI Rats

1. Hindlimb Functional Restoration

Sprague-Dawley (SD) rats were obtained from the Animal Center of National Yang-Ming University or National Science Council, Taiwan. Female adult SD rats ranging from 240-280 g were used for induction of contusive SCI models. Animal handling and experimental protocols were carefully reviewed and approved by the animal studies committee of Taipei Veteran General Hospital.

Contusive SCI rats were induced using the NYU weight-drop device. Female adult Sprague-Dawley rats were anesthetized. Dorsal laminectomy was carried out at the level of the ninth thoracic (T) vertebra. The dorsal surface of T9-T10 spinal cord was injured by dropping a 10 g rod from a height of 50 mm. The dura was left mechanically intact, while weight-drop injury leads to the characteristic of egg-shaped zone of necrosis, extending several spinal cord segments rostrocaudally (Grossman et al., 2001; Widenfalk et al., 2001).

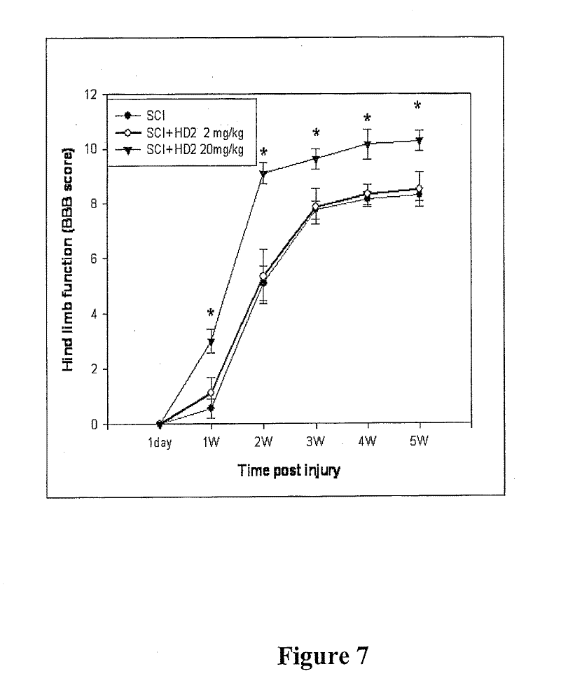

Thirty minutes prior to eliciting severe SCI, adult rats were intraperitoneally injected with 2 or 20 mg/kg of HD2 based on the weight of the rats. After SCI, the rats were daily administrated by HD2 in an amount of 2 or 20 mg/kg/day for 7 consecutive days. The hindlimb performance of the rats was monitored weekly post-injury (up to 5 weeks) using the open field locomotor test (BBB scale) (Basso et al., J. Neurotrauma 12 (1995): 1-21; Basso et al., Exp. Neurol. 139 (1996): 244-256). Two observers, unaware of the experimental procedures, performed the evaluation weekly according to the BBB scale, which ranges from 0 (no hindlimb movement) to 21 (normal movement-coordinated gait). FIG. 7 shows the evaluation result of the hindlimb performance for the SCI rats after HD2 treatment.

As shown in FIG. 7, intraperitoneal administration of HD2 (20 mg/kg) significantly facilitated functional recovery of the SCI rats. This also means that HD2 can cross the blood-spinal cord-barrier.

In addition, at 5 weeks post-injury, rats were sacrificed and perfused intravascularly with 4% paraformaldehyde. The thoracic regions of the spinal cords were then sagittally sectioned (10 μm-thick) and processed for immunohistochemical staining with anti-neurofilament antibodies (for neurons).

According to the result of the immunohistochemical staining (data not shown), the spinal axons (neurofilament-positive) were better preserved in the HD2 (20 mg/kg)-treated SCI section than in the control SCI section.

2. Early Microglial Infiltration

Traumatic spinal cord injury is devastating and initiates a series of cellular and molecular events that include both primary and secondary injury cascades. Injury to the spinal cord provokes an inflammatory reaction that initially results in further tissue damage. Attenuation of the early inflammatory response to spinal cord injury may therefore limit the extent of tissue injury, and accordingly, the consequent disability.

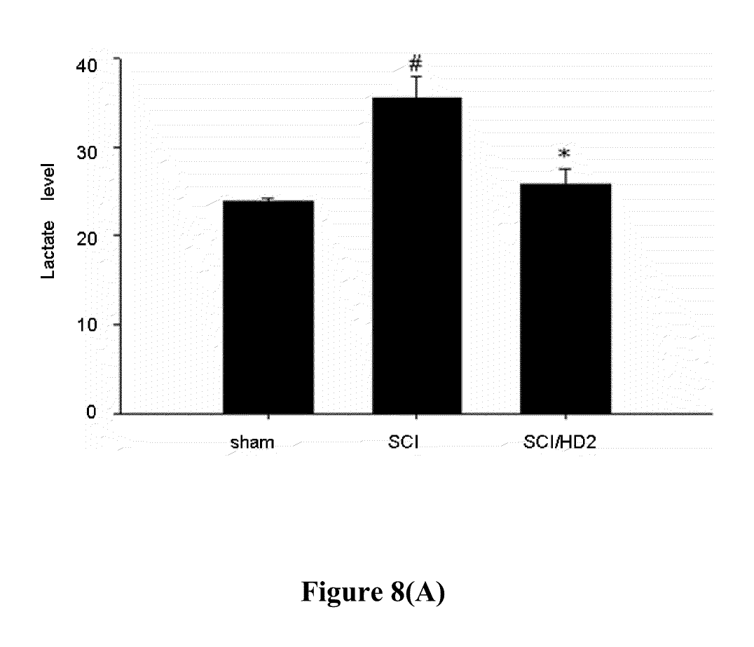

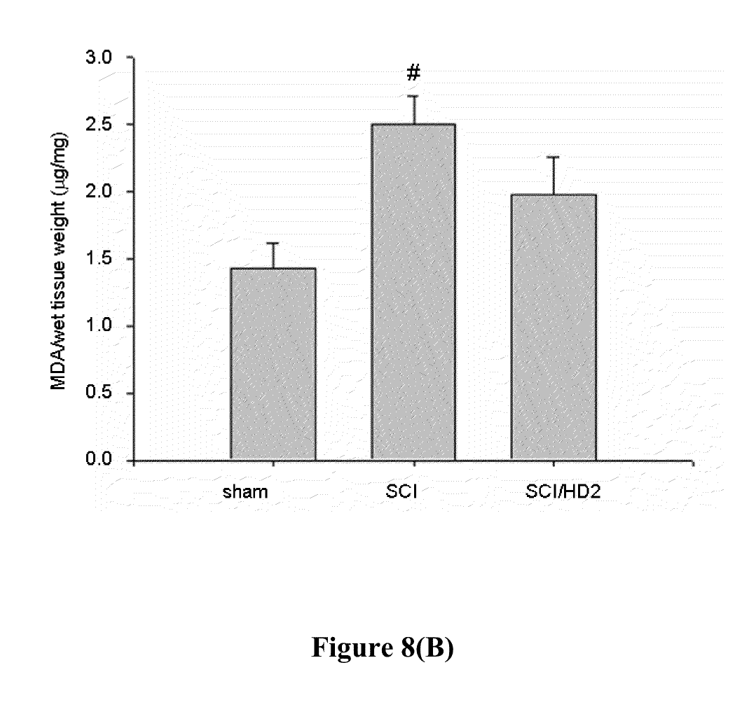

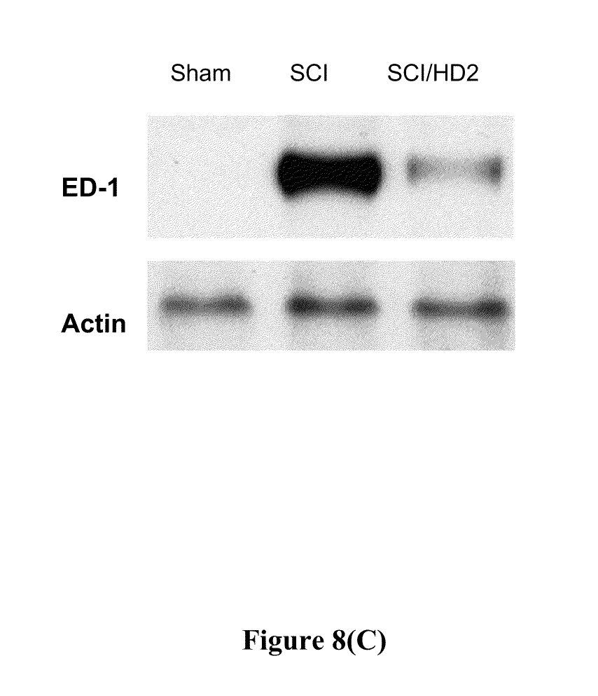

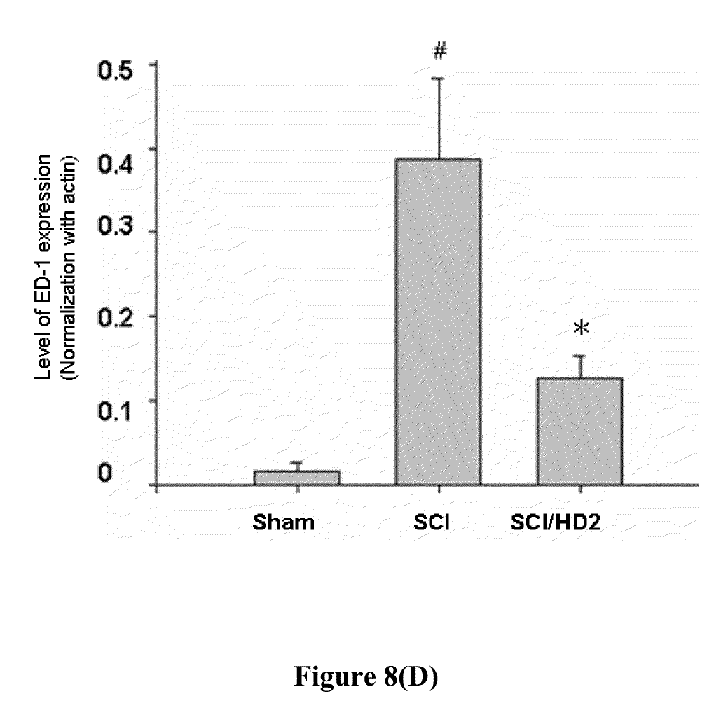

SCI rats with or without intraperitoneal administration of HD2 (20 mg/kg) were sacrificed at the third day post-injury. The injured epicenter of thoracic spinal cords (about 1.5 cm) was rapidly removed and longitudinal dissected into equal 2 segments. One half of the cords was homogenized in ice-cold PBS buffer containing 5 mM BHT by sonication (Probe-tip sonicator, MISONIX) and proceeded for measurement of lipid peroxidation malondialdehyde (MDA) (Bioxytech LPO-586) or lactate level. FIGS. 8 (A) and (B) show the results of the measurement.

On the other hand, the other half of the cords was homogenized in ice-cold extraction buffer (7 M urea, 2 M thiourea, 4% CHAPS, and 40 mM Tris buffer (pH7.5) and protease inhibitors (Roche 11836145001), and then subject to western blot analysis. Briefly, protein concentration was determined by the Bradford method (Bio-Rad Protein Assay, Bio-Rad Laboratories). Equal amounts of proteins were loaded and separated on 8% SDS-PAGE gels. After transfer, the resulted PVDF membrane was probed with anti-ED-1 and anti-actin antibodies. FIG. 8(C) shows the results of the western blotting. FIG. 8 (D) is the quantification of the cell expression.

As shown in FIGS. 8 (A) and (B), injury to the spinal cord provoked an increase of lactate level and MDA level, indicating impairment in energy metabolism and free radical damage, respectively; and intraperitoneal injection of HD2 (20 mg/kg) to SCI rats for three days reduced both of the lactate and MDA levels.

Consistently, as shown in FIGS. 8 (C) and (D), SCI induced protein expression of ED-1, indicating increased infiltration of activated microglia; and HD2 effectively reduced the microglia infiltration (20 mg/kg; 3 ip injections).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for treating neural damage, comprising administering to a subject in need thereof an effective amount of a composition containing an aqueous extract of bupleurum roots and an aqueous extract of scutellaria roots.

2. The method of claim 1, wherein the bupleurum roots for preparing the aqueous extract thereof and the scutellaria roots for preparing the aqueous extract thereof are at a weight ratio of 7:3.

3. The method of claim 1, wherein the aqueous extract of bupleurum roots is prepared from roots of Bupleurum scorzonerifolfium Wild and the aqueous extract of scutellaria roots is prepared from roots of Scutellaria baicalensis Georgi.

4. The method of claim 1, wherein the composition is prepared by boiling bupleurum roots and scutellaria roots together in water to obtain a mixture, filtering the mixture to obtain a solution, and lyophilizing the solution to obtain a powder.

5. The method of claim 4, wherein the bupleurum roots and scutellaria roots are at a weight ratio of 7:3.

6. The method of claim 1, wherein the composition comprises baicalin, baicalein, wogonin, wogonoside, saikosaponin A, saikosaponin C, and saikosaponin D.

7. The method of claim 4, wherein the powder is dissolved in saline immediately before administration.

8. The method of claim 1, wherein the effective amount of the composition is 20 mg per kg of the subject.

9. The method of claim 1, wherein the subject suffers from spinal cord injury (SCI), traumatic brain injury, peripheral nerve injury, Parkinson's disease, Huntington's disease, Alzheimer's disease, amyotrophic lateral sclerosis (ALS), epileptic seizure, or brain ischemia.

10. The method of claim 9, wherein the subject suffers from SCI.

11. The method of claim 9, wherein the SCI is contusive SCI.

12. The method of claim 10, wherein the bupleurum roots for preparing the aqueous extract thereof and the scutellaria roots for preparing the aqueous extract thereof are at a weight ratio of 7:3.

13. The method of claim 10, wherein the aqueous extract of bupleurum roots is prepared from roots of Bupleurum scorzonerifolfium Wild and the aqueous extract of scutellaria roots is prepared from roots of Scutellaria baicalensis Georgi.

14. The method of claim 10, wherein the composition is prepared by boiling bupleurum roots and scutellaria roots together in water to obtain a mixture, filtering the mixture to obtain a solution, and lyophilizing the solution to obtain a powder.

15. The method of claim 14, wherein the powder is dissolved in saline immediately before administration.

16. The method of claim 10, wherein the composition comprises baicalin, baicalein, wogonin, wogonoside, saikosaponin A, saikosaponin C, and saikosaponin D.

17. The method of claim 10, wherein the effective amount of the composition is 20 mg per kg of the subject.
